(19) United States
(12) Patent Application Publication
(10) Pub. No.: US 2003/0158264 A1
(43) Pub. Date: Aug. 21, 2003

(54) Orally administrable pharmaceutical formulation comprising ephedrine hydrochloride and process for preparing the same

(76) Inventors: Ramachandran Radhakrishnan, Bangalore (IN); Nehru Babu Gaddipati, Somerset, NJ (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/096,708

(22) Filed: Mar. 13, 2002

(30) Foreign Application Priority Data
Feb. 20, 2002 (IN) 128/DEL/2002

(51) Int. Cl. A61K 31/137; A61K 9/48

(52) U.S. Cl. 514/649; 424/452

(57) ABSTRACT

Disclosed is a pharmaceutical formulation for oral administration through a soft gelatin capsule drug delivery device, wherein the pharmaceutical formulation includes Ephedrine HCl and an expectorant as active ingredients. Preferred formulations include Ephedrine embedded into an oily matrix, an expectorant; a surfactant; a suspending agent; and a suspension medium, wherein the expectorant is guaifenesin, the surfactant is lecithin, the suspending agent is yellow beeswax, and the suspension medium is soybean oil. A preferred formulation consists essentially of either about 25 mg or about 12.5 mg by weight of Ephedrine HCl, about 200 mg by weight of guaifenesin, about 0.1-5.0 mg by weight of yellow beeswax, about 10-15 mg by weight of lecithin; and about 200-300 mg by weight of soybean oil. Also disclosed is a process for preparing the formulation.

### BACKGROUND OF THE INVENTION

1. Field of the Invention

This invention in general relates to orally administrable pharmaceutical formulations and in particular to a pharmaceutical formulation prepared into a soft gelatin capsule containing Ephedrine hydrochloride as one of its active ingredients.

2. Description of the Related Art

Ephedrine hydrochloride is a drug that has serious potential for abuse. This is so because Ephedrine can be extracted from various drug products containing Ephedrine hydrochloride and can be converted into amphetamines. Amphetamines have potentially lethal stimulant effects on the central nervous system and heart and it is therefore useful to minimize such abuse potential.

Ephedrine HCl is well known as a vasoconstrictor. Its use is therefore significant in symptomatic relief from congestion occurring in bronchial asthma. Ephedrine as a broncho-dilator has a slower onset but longer duration of action. Ephedrine provides temporary relief from shortness of breath, tightness of chest and wheezing in bronchial asthma.

Pharmaceutical compositions comprising Ephedrine HCl as its principal ingredient is known. U.S. Pat. No. 5,858,371 to Singh et al. describes a vasoconstrictor used in the composition as Ephedrine. This disclosure is directed to a pharmaceutical composition for topical application.

U.S. Pat. No. 6,027,746 to Lech et. al describes a liquid oral suspension incorporated into a softgel capsule wherein the decongestant is selected from a group which includes Ephedrine. The formulation also includes an active agent consisting of a particulate adsorbate. The drug delivery device is in chewable form.

A composition including soybean oil, yellow beeswax and lecithin has been disclosed in U.S. Pat. No. 6,309,667 to Horvath et. al. This disclosure is not directed to Ephedrine HCl as an ingredient in combination with the other excipients.

U.S. Pat. No. 5,175,002 is directed to a suspension formulation comprising soybean oil, lecithin and wax. However the active ingredient in this formulation is Amantidine hydrochloride.

### SUMMARY OF THE INVENTION

It has been found that patient compliance is improved if a soft gelatin capsule is used for drug administration, because of its soft, elastic character which makes it easier to swallow when compared to conventional tablets or hard gelatin capsules. Furthermore, since the dosage form is generally swallowed without chewing, it is unnecessary to flavor or otherwise mask any unpleasant taste of the active pharmaceutical ingredients. Finally, unlike tablets, soft gelatin capsules do not chip or powder. Accordingly we sought to devise a soft gelatin capsule formulation of Ephedrine HCl because of these and other reasons.

In accordance with one preferred embodiment there is provided an orally administrable pharmaceutical formulation consisting essentially of an active pharmaceutical ingredient embedded into an oily matrix; an expectorant; a surfactant; a suspending agent; and a suspension medium.

In accordance with one preferred embodiment there are provided soft gelatin capsules of a pharmaceutical formulation consisting essentially of about 25 mg by weight of Ephedrine HCl, about 200 mg by weight of guaifenesin, about 0.1-5.0 mg by weight of yellow beeswax, about 10-15 mg by weight of lecithin and about 200-300 mg by weight of soybean oil.

In accordance with another preferred embodiment there are provided soft gelatin capsules of a pharmaceutical formulation consisting essentially of about 12.5 mg by weight of Ephedrine HCl, about 200 mg by weight guaifenesin, about 0.1-5.0 mg by weight of yellow beeswax, about 10-15 mg by weight of lecithin and about 200-300 mg by weight of soybean oil.

In accordance with another preferred embodiment there are provided methods of making a pharmaceutical formulation comprising the steps of preparing an oily matrix consisting of soybean oil and beeswax, blending lecithin to said oily matrix, adding guaifenesin to said matrix, mixing an active pharmaceutical ingredient into the matrix and enclosing the oily matrix embedded pharmaceutical complex into a capsule, wherein the formulation preferably comprises about 25 mg by weight of Ephedrine HCl as the active pharmaceutical ingredient, about 200 mg by weight guaifenesin, about 0.1-5.0 mg by weight of yellow beeswax, about 10-15 mg by weight of lecithin and about 200-300 mg by weight of soybean oil. In a preferred embodiment the capsule is a soft gelatin capsule drug delivery device.

In accordance with another preferred embodiment there is provided a method of making soft gelatin capsules of a pharmaceutical formulation consisting essentially of about 12.5 mg by weight of Ephedrine HCl, about 200 mg by weight guaifenesin, about 0.1-5.0 mg by weight of yellow beeswax, about 10-15 mg by weight of lecithin and about 200-300 mg by weight of soybean oil.

One possible advantage of preferred embodiments is that the Ephedrine (alone or together with one or more other components) is coated with wax, making the possible extraction of Ephedrine and its derivatives more difficult. Another possible advantage of preferred embodiments is that the drug delivery of the pharmaceutical formulation is achieved by a soft gelatin capsule and this makes it relatively difficult for someone to extract the active, unlike the case of a tablet as an OTC drug product. Hence the possibility of abuse of the drug is minimized.

In yet another advantage, preferred formulations include guaifenesin in combination with Ephedrine HCl. This enables the composition to ease breathing for bronchial muscles and helps loosen phlegm and thin bronchial secretions to rid bronchial passageways of bothersome mucus and make coughs more productive.

Another possible advantage of preferred embodiments is that preferred formulations include excipients like yellow beeswax and soybean oil, which are natural substances that make the extraction of ephedrine more difficult. This, in conjunction with the soft gelatin encapsulation makes it relatively a complex multi step process to extract amphetamines from the oily matrix. Thus the preferred embodiments considerably minimize the potential to abuse the drug product.

### DETAILED DESCRIPTION OF THE PREFERRED EMBODIMENTS

The present invention relates to pharmaceutical formulations comprising Ephedrine HCl for oral administration in the form of soft gelatin capsules. Preferred formulations also include guaifenesin, yellow beeswax, soybean oil and lecithin. In a preferred embodiment, the formulation consists essentially of the foregoing materials. In preferred embodiments we have used soybean oil as a suspension medium and yellow beeswax as a suspending agent.

Preferred formulations include guaifenesin that promotes lower respiratory tract drainage by thinning bronchial secretions, lubricates irritated respiratory track membranes through increased mucous flow and facilitates removal of viscous, inspissated mucus. As a result the sinus and bronchial drainage is improved and dry non-productive coughs become more productive and less frequent.

According to a preferred embodiment, wax forms part of the fill composition that is inside the gelatin shell. The wax and oil mixture makes it difficult to isolate the active from the formulation.

The following examples illustrate preferred embodiments of pharmaceutical compositions comprising Ephedrine HCl as principal ingredient.

### EXAMPLES

### Example 1

| | |
|---|---|
| Ingredients | Composition by weight |

| | | |
|---|---|---|
| Ephedrine HCl, USP | 25 | mg |
| Guaifenesin, USP | 200 | mg |
| Yellow Beeswax | 0.1-5.0 | mg |
| Lecithin, NF | 10-15 | mg |
| Soybean Oil, USP | 200-300 | mg |

### Example 2

| | |
|---|---|
| Ingredients | Composition by weight |

| | | |
|---|---|---|
| Ephedrine HCl, USP | 12.5 | mg |
| Guaifenesin, USP | 200 | mg |
| Yellow Beeswax | 0.1-5.0 | mg |
| Lecithin, NF | 10-15 | mg |
| Soybena Oil, USP | 200-300 | mg |

Although ephedrine HCl is a preferred form of the ephedrine, use of the free base or other salts of ephedrine is also contemplated.

In general, gelatin capsule formulations for soft gelatin capsule comprise raw gelatin, plasticizer, solvent and optional ingredients such as flavors and colorants. Typically the plasticizer includes glycerin or sorbitol. A preferred plasticizer in this case is glycerin. One preferred gelatin formulation for the soft gelatin capsule used in accordance with preferred embodiments includes gelatin in the range of about 40-45 % and a plasticizer in the range of about 18-25 %. Capsule formulation can also include other suitable additives, which impart specific characteristics such as the look and feel of the capsule.

The following examples illustrate preferred embodiments of several soft-gelatin-shell Ephedrine HCl/Guaifenesin formulations. These examples illustrate particular embodiments of the invention and are not intended to limit the scope of the invention in any way.

### Example 3

| Ingredient | Percentage by weight |
|---|---|
| Gelatin | 43.4% |
| Glycerin | 20.0% |
| Water | 36.6% |

### Example 4

| Ingredient | Percentage by weight |
|---|---|
| Gelatin | 58.5% |
| Glycerine | 31.5% |
| Water | 10.0% |

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the formulations and methods may be formulated or performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

What is claimed is:

1. An orally administrable pharmaceutical formulation consisting essentially of an active pharmaceutical ingredient embedded into an oily matrix; an expectorant; a surfactant; a suspending agent; and a suspension medium.

2. The orally administrable pharmaceutical formulation according to claim 1, wherein the active pharmaceutical ingredient is Ephedrine hydrochloride.

3. The orally administrable pharmaceutical formulation according to claim 1, wherein the expectorant is guaifenesin.

4. The orally administrable pharmaceutical formulation according to claim 1, wherein the surfactant is lecithin.

5. The orally administrable pharmaceutical formulation according to claim 1, wherein the suspending agent is yellow beeswax.

6. The orally administrable pharmaceutical formulation according to claim 1, wherein the suspension medium is soybean oil.

7. An orally administrable pharmaceutical formulation consisting essentially of:
about 25 mg of Ephedrine HCl,
about 200 mg of guaifenesin,
about 0.1-5.0 mg of yellow beeswax,
about 10-15 mg of lecithin; and
about 200-300 mg of soybean oil.

8. The orally administrable pharmaceutical formulation according to claim 7, wherein the lecithin is employed to provide lubricity to the matrix.

9. The orally administrable pharmaceutical formulation according to claim 7, wherein the oily matrix-embedded active pharmaceutical ingredients is disposed into a capsule.

10. The orally administrable pharmaceutical formulation according to claim 9, wherein the capsule is a soft gelatin capsule.

11. An orally administrable pharmaceutical formulation consisting essentially of:
about 12.5 mg of Ephedrine HCl,
about 200 mg of guaifenesin,
about 0.1-0.5 mg of yellow beeswax,
about 10-15 mg of lecithin; and
about 200-300 mg of soybean oil.

12. The orally administrable pharmaceutical formulation according to claim 11, wherein the lecithin is employed to provide lubricity to the matrix.

13. The orally administrable pharmaceutical formulation according to claim 11, wherein the oily matrix-embedded active pharmaceutical ingredients is disposed into a capsule.

14. The orally administrable pharmaceutical formulation according to claim 13, wherein the capsule is a soft gelatin capsule.

15. A process for preparing of an orally administrable pharmaceutical formulation comprising
preparing an oily matrix consisting of soybean oil and beeswax;
blending lecithin to said oily matrix;
adding guaifenesin to said matrix;
mixing an active pharmaceutical ingredient into said matrix; and
encapsulating the oily matrix-embedded pharmaceutical complex into a capsule.

16. The process for preparing of an orally administrable pharmaceutical formulation according to claim 15, wherein the active pharmaceutical ingredient is Ephedrine Hydrochloride.

17. The process for preparing of an orally administrable pharmaceutical formulation according to claim 15, wherein the capsule is a soft gelatin capsule.
